(19) United States
(12) Patent Application Publication
(10) Pub. No.: US 2014/0219962 A1
(43) Pub. Date: Aug. 7, 2014

(54) POROUS CHITOSAN SCAFFOLDS AND RELATED METHODS

(71) Applicant: University of Washington through its Center for Commercialization, Seattle, WA (US)

(72) Inventors: Miqin Zhang, Bothell, WA (US); Soumen Jana, Seattle, WA (US)

(73) Assignee: University of Washington through its Center for Commercialization, Seattle, WA (US)

(21) Appl. No.: 14/169,020

(22) Filed: Jan. 30, 2014

Related U.S. Application Data
(60) Provisional application No. 61/759,636, filed on Feb. 1, 2013.

(51) Int. Cl. A61K 9/70; A61K 31/722

(52) U.S. Cl. CPC A61K 9/70; A61K 31/722
USPC 424/93.1; 536/20; 514/55; 435/375

(57) ABSTRACT

Porous chitosan scaffold having high mechanical strength, methods for making the porous chitosan scaffold, and method for using the porous chitosan scaffold.

### CROSS-REFERENCE TO RELATED APPLICATION

This application claims the benefit of U.S. Patent Application No. 61/759,636, filed Feb. 1, 2013, incorporated herein by reference in its entirety

### STATEMENT REGARDING SEQUENCE LISTING

The sequence listing associated with this application is provided in text format in lieu of a paper copy and is hereby incorporated by reference into the specification. The name of the text file containing the sequence listing is 43570_Seq_Final_{—}20140123.txt. The text file is 2.72 KB; was created on Jan. 23, 2014; and is being submitted via EFS-Web with the filing of the specification.

### BACKGROUND OF THE INVENTION

Chitosan, a widely used natural cationic polymer, has recently drawn considerable attention in biomedicine. Commonly produced from crab shells, chitosan is non-toxic and biodegradable, and its hydrophilic surface promotes cell adhesion and proliferation. Unlike the natural polymers derived from costly mammalian proteins, chitosan evokes minimal foreign-body response and fibrous encapsulation, and has unlimited material sources and may be produced with excellent reproducibility. Chitosan is particularly attractive for bone tissue engineering due to its high osteoconductivity that promotes bone growth both in vitro and in vivo and its ability to accelerate osteogenic differentiation.

One major limitation of chitosan is its low mechanical strength, which precludes pristine chitosan scaffolds for load-bearing usage. Reported compressive modulus and strength of chitosan scaffolds differ vastly, but fall in the ranges of 0.0038-2.56 MPa and 0.059-0.125 MPa, respectively, which are significantly lower than the compressive strength and modulus of cancellous bone, which are in the ranges of 9-20 MPa and 0.5-10 MPa, respectively. An extensive effort has been made to develop chitosan-based scaffolds with improved mechanical strength, mostly through the addition of a reinforcement agent (e.g., beta-tricalcium phosphate, hydroxyapatite), incorporation of a synthetic polymer (e.g., poly methyl-methacrylate, poly-L-lactic acid), or complexing with another polymer. These chitosan-based composite scaffolds have significantly-increased mechanical strength and modulus (up to 10 MPa and 0.5 MPa respectively) compared to pristine chitosan scaffolds. However, despite their impressive strengths, such scaffolds are not without limitations. If the concentration of the additive is low, no substantial improvement in mechanical properties can be achieved, whereas with a higher concentration of the additive (e.g., >30 wt %), improved mechanical properties are obtained at the cost of compromised structure and porosity. Yet, a high concentration of additives can potentially alter biological properties of the chitosan scaffold, which may be undesirable for the intended application.

Despite the advances in the development of chitosan-based scaffolds noted above for biomedical applications and tissue engineering, a need exists for a chitosan scaffold that maintains the advantageous properties of chitosan and addresses the disadvantages associated with the weak mechanical strength of scaffolds produced from chitosan. The present invention seeks to fulfill this need and provides further related advantages.

### SUMMARY OF THE INVENTION

The present invention provides porous chitosan scaffolds, methods for using the porous chitosan scaffolds, and methods for making the porous chitosan scaffolds.

In one aspect, the invention provides porous chitosan scaffolds.

In one embodiment, the porous chitosan scaffold comprises chitosan and has a compressive modulus from about 3 to about 20 MPa and compressive strength from about 0.2 to about 2.0 MPa. The scaffold has pores having pore walls.

In another embodiment, the porous chitosan scaffold consists essentially of chitosan. The scaffold has pores having pore walls. In certain of these embodiments, the scaffold has a compressive modulus from about 3 to about 20 MPa and compressive strength from about 0.2 to about 2.0 MPa.

In a further embodiment, the porous chitosan scaffold consists of chitosan. The scaffold has pores having pore walls. In certain of these embodiments, the scaffold has a compressive modulus from about 3 to about 20 MPa and compressive strength from about 0.2 to about 2.0 MPa.

In certain embodiments, the scaffolds have a porosity of from about 88 to about 98 percent. In certain embodiments, the scaffolds have a pore size distribution from about 30 to about 500 μm. In certain embodiments, the scaffolds' pore walls have a thickness from about 12 to about 50 μm.

In certain embodiments, the scaffolds further include an additive for supporting cell growth. In certain embodiments, the scaffolds further include cells.

In another aspect, the invention provides a method for expanding a population of or culturing cells.

In one embodiment, the method includes seeding a porous scaffold of the invention with cells, and culturing the cells in the presence of the scaffold to provide a scaffold populated with the cells.

In another embodiment, the invention provides a method for tissue engineering. In the method a scaffold of the invention is introduced into a tissue to be engineered.

In a further aspect of the invention, methods for making a porous chitosan scaffold is provided.

In one embodiment, the method includes:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity, and wherein the second aqueous chitosan solution does not include an organic solvent;

(c) freezing the second aqueous chitosan solution to provide a frozen chitosan composition; and

(d) freeze-drying the frozen chitosan composition to provide a porous chitosan scaffold.

In another embodiment, the method for making the porous chitosan scaffold consists essentially of:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity;

(c) freezing the second aqueous chitosan solution to provide a frozen chitosan composition; and

(d) freeze-drying the frozen chitosan composition to provide a porous chitosan scaffold.

In a further embodiment, the method for making the porous chitosan scaffold consists of:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity;

(c) freezing the second aqueous chitosan solution to provide a frozen chitosan composition; and

(d) freeze-drying the frozen chitosan composition to provide a porous chitosan scaffold.

In certain embodiments of the methods, the acid is a weak acid. Representative weak acids include acetic acid, lactic acid, formic acid, and mixtures thereof. In other embodiments, the acid is a strong acid. Representative strong acids include hydrochloric acid, sulfuric acid, nitric acid, phosphoric acid, and mixtures thereof.

In certain embodiments, the concentration of chitosan in the first aqueous chitosan solution is from about 2 to about 12 weight percent.

In certain embodiments, conditioning the first aqueous chitosan solution comprises maintaining the solution at a temperature from about 4 to about 30° C. for 12 to 48 hours.

In certain embodiments of the methods, the second viscosity is about 25% greater than the first viscosity. In other embodiments, the second viscosity is about 50% greater than the first viscosity. In further embodiments, the second viscosity is about 75% greater than the first viscosity. In certain embodiments, the second viscosity is the equilibrium viscosity.

In certain embodiments, freezing the second aqueous chitosan solution comprises freezing at a temperature of from about −196 to about 0° C. for 12 to 48 hours.

In certain embodiments, freeze-drying the frozen chitosan composition comprises freeze drying under reduced pressure at a temperature of from about −90 to about −5° C.

In certain embodiments, the methods of the invention provide a porous chitosan scaffold having a compressive modulus from about 3 to about 20 MPa and compressive strength from about 0.2 to about 2.0 MPa.

In certain embodiments, the methods of the invention provide a porous chitosan scaffold consisting essentially of chitosan. In other embodiments, the methods of the invention provide a porous chitosan scaffold consisting of chitosan.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A compares viscosity of 6 wt % chitosan gel-solutions measured at 4 hr intervals from the onset of dissolution to 24 hrs.

FIG. 1B compares viscosity of 6 wt % chitosan gel-solutions measured at 24 and 28 hr time points from the onset of dissolution.

FIG. 2A is a scanning electron microscope (SEM) image of a representative chitosan scaffold of the invention prepared from a solution with a chitosan concentration of 4 wt %. Scale bar represents 100 μm.

FIG. 2B is an SEM image of a representative chitosan scaffold of the invention prepared from a solution with a chitosan concentration of 6 wt %. Scale bar represents 100 μm.

FIG. 2C is an SEM image of a representative chitosan scaffold of the invention prepared from a solution with a chitosan concentration of 8 wt %. Scale bar represents 100 μm.

FIG. 2D is an SEM image of a representative chitosan scaffold of the invention prepared from a solution with a chitosan concentration of 12 wt %. Scale bar represents 100 μm.

FIG. 3A are photographs of chitosan discs illustrating shape retention capability of chitosan scaffolds prepared at chitosan concentrations of 4, 6, 8, 12 wt % (from top to bottom). Shown in the figure are scaffolds in dry state, and after soaked for 2 weeks in PBS, DMEM (CM), and SBF solutions.

FIG. 3B compares swelling ratios of chitosan scaffolds prepared from solutions of different chitosan concentrations (*p<0.05 by student's t-test, n=3, and all values within brackets were statistically significantly different from each other).

FIG. 4A is a graph illustrating stress-strain relationships of representative chitosan scaffolds prepared at different chitosan concentrations and acquired in compression tests.

FIG. 4B is a graph illustrating stress-strain relationships of representative chitosan scaffolds prepared at different chitosan concentrations and acquired in tensile tests.

FIG. 5A illustrates the crystalline properties of chitosan characterized by XRD. Chitosan samples were prepared from solutions of different chitosan concentrations but same acid concentration (0.34M).

FIG. 5B illustrates the crystalline properties of chitosan characterized by XRD. Chitosan samples were prepared from solutions of the same chitosan concentration (4 wt %) but different acetic acid concentrations (1M and 0.34M).

FIG. 6 compares the proliferation of MG-63 cells on chitosan scaffolds prepared from solutions of different chitosan concentrations (or different mechanical strength) over a 7-day period, assessed by the Alamar Blue assay. Data refer to mean value±standard deviation (*p<0.05, by student's t-test, n=3, two numbers bracketed were statistically significantly different).

FIG. 7A is an SEM image of MG-63 grown in a representative chitosan scaffold of the invention after 7 days of culture. The scaffold was prepared from a solution with a chitosan concentration of 4 wt %. Scale bar represents 100 μm.

FIG. 7B is an SEM image of MG-63 grown in a representative chitosan scaffold of the invention after 7 days of culture. The scaffold was prepared from a solution with a chitosan concentration of 6 wt %. Scale bar represents 100 μm.

FIG. 7C is an SEM image of MG-63 grown in a representative chitosan scaffold of the invention after 7 days of culture. The scaffold was prepared from a solution with a chitosan concentration of 8 wt %. Scale bar represents 100 μm.

FIG. 7D is an SEM image of MG-63 grown in a representative chitosan scaffold of the invention after 7 days of culture. The scaffold was prepared from a solution with a chitosan concentration of 12 wt %. Scale bar represents 100 μm.

FIG. 8A illustrates cell morphology observed by SEM on a representative chitosan scaffold prepared from a solution with a chitosan concentration of 4 wt %. Scale bar represents 10 μm.

FIG. 8B illustrates cell morphology observed by SEM on a representative chitosan scaffold prepared from a solution with a chitosan concentration of 6 wt %. Scale bar represents 10 μm.

FIG. 8C illustrates cell morphology observed by SEM on a representative chitosan scaffold prepared from a solution with a chitosan concentration of 8 wt %. Scale bar represents 10 μm.

FIG. 8D illustrates cell morphology observed by SEM on a representative chitosan scaffold prepared from a solution with a chitosan concentration of 12 wt %. Scale bar represents 10 μm.

FIG. 9A is a fluorescence image of osteoblast cells cultured on a representative chitosan scaffold prepared from a solution with a chitosan concentration of 4 wt %. Osteocalcin is stained green and nuclei stained blue. Scale bar represents 20 μm.

FIG. 9B is a fluorescence image of osteoblast cells cultured on a representative chitosan scaffold prepared from a solution with a chitosan concentration of 6 wt %. Osteocalcin is stained green and nuclei stained blue. Scale bar represents 20 μm.

FIG. 9C is a fluorescence image of osteoblast cells cultured on a representative chitosan scaffold prepared from a solution with a chitosan concentration of 8 wt %. Osteocalcin is stained green and nuclei stained blue. Scale bar represents 20 μm.

FIG. 9D is a fluorescence image of osteoblast cells cultured on a representative chitosan scaffold prepared from a solution with a chitosan concentration of 12 wt %. Osteocalcin is stained green and nuclei stained blue. Scale bar represents 20 μm.

FIG. 10A compares osteogenic activity (relative osteocalcin expression by immunoblot) of MG-63 cells cultured on representative chitosan scaffolds of the invention for 7 days. Values presented are normalized to relative expression in 4 wt % scaffolds.

FIG. 10B compares osteogenic activity (relative osteocalcin expression by RT-PCR) of MG-63 cells cultured on representative chitosan scaffolds of the invention for 7 days. Values presented are normalized to relative expression in 4 wt % scaffolds.

FIG. 10C illustrates osteocalcin expression by flow cytometry. Data refer to mean value±standard deviation (*p<0.05, by student t-test, n=3, two values bracketed were statistically significantly different).

### DETAILED DESCRIPTION OF THE INVENTION

The present invention provides high-strength porous chitosan scaffolds to suit a wide range of biomedical uses and methods for producing the chitosan scaffolds that control the scaffolds' mechanical properties.

Porous Chitosan Scaffolds

In one aspect, the invention provides porous chitosan scaffolds. The porous scaffolds have improved mechanical strength compared to porous scaffolds known in the art prepared from chitosan.

In one embodiment, the porous chitosan scaffold has a compressive modulus from about 3 to about 20 MPa and compressive strength from about 0.2 to about 2.0 MPa. The chitosan scaffold includes pores and has pore walls. In certain embodiments, the compressive modulus is from about from about 5 about 15 MPa. In certain embodiments, the compressive strength is from about 0.3 to about 1.8 MPa.

In another embodiment, the porous chitosan scaffold consists essentially of chitosan. The chitosan scaffold includes pores and has pore walls. In certain embodiments, the scaffold has a compressive modulus from about 3 to about 20 MPa and compressive strength from about 0.2 to about 2.0 MPa.

In a further embodiment, the porous chitosan scaffold consists of chitosan. The chitosan scaffold includes pores and has pore walls. In certain embodiments, the scaffold has a compressive modulus from about 3 to about 20 MPa and compressive strength from about 0.2 to about 2.0 MPa.

The scaffolds of the invention have a porosity of from about 88 to about 98 percent and a pore size distribution from about 30 to about 500 μm. For bone tissue engineering, scaffolds may have a pore size up to about 500 μm. For skeletal muscle tissue engineering, scaffolds may have a pore size from about 100 to about 200 μm.

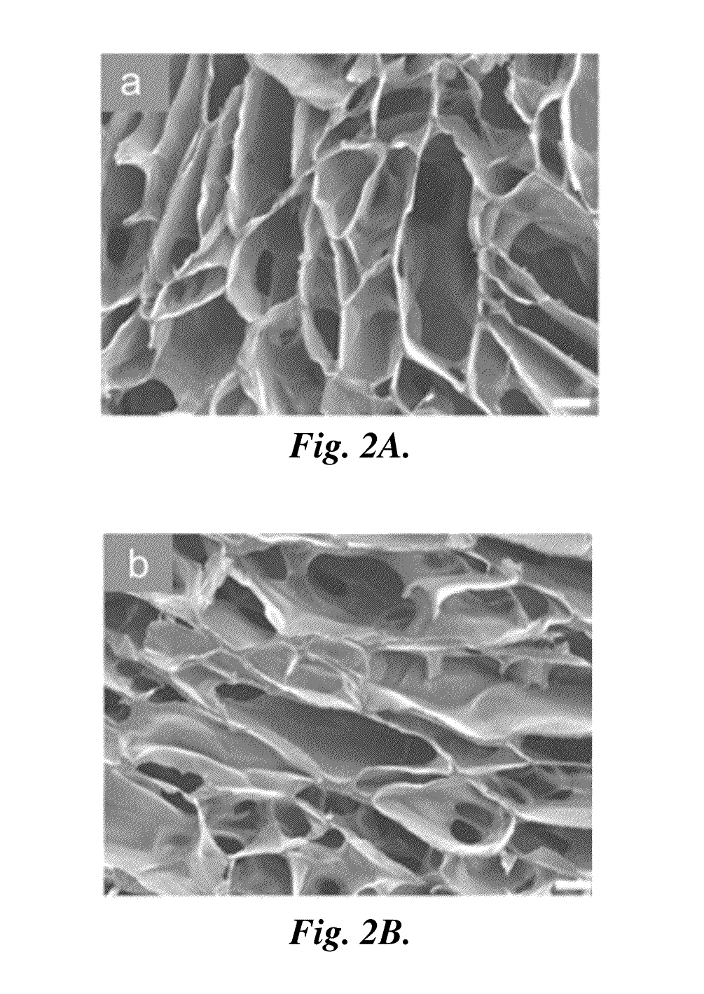

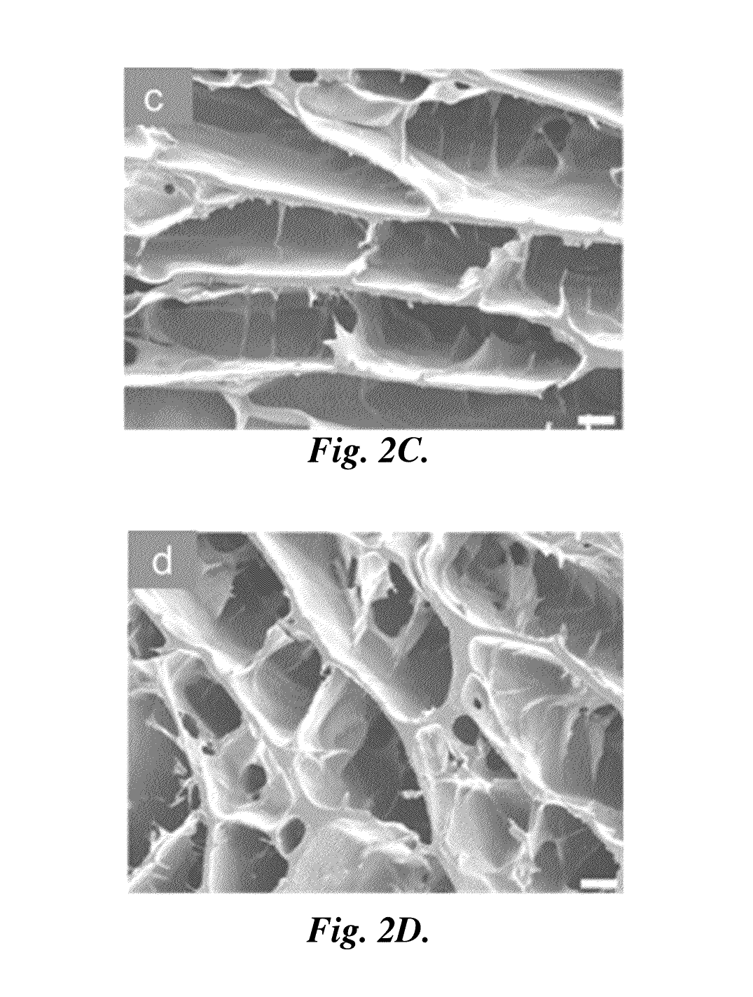

The scaffolds of the invention have includes pores and pore walls that have a thickness from about 12 to about 50 μm. In certain embodiments, the pore walls have a thickness from about 20 to about 40 μm. Images of representative scaffolds of the invention illustrating pores and pore wall thick ness are shown in FIGS. 2A-2D.

The scaffolds of the invention can be prepared from chitosan having a weight average molar mass from about 5,000 to about 500,000. In certain embodiments, the chitosan has a weight average molar mass from about 20,000 to about 200,000. Chitosan having a range of deacetylation is suitable for making the scaffolds. In certain embodiments, the chitosan is from about 60 to about 100% deacetylated. In other embodiments, the chitosan is from about 85 to about 95% deacetylated.

For cell culture and tissue engineering uses, the scaffolds of the invention may further include one or more additives for supporting cell growth. Representative additives include cell growth factors.

For cell culture and tissue engineering uses, cells can be added to the scaffolds of the invention. The type of the cells that can be added to the scaffolds is not critical. Representative cells for tissue engineering include bone, cartilage, epithelial, nerve, blood, tendon, muscle, liver, lung, kidney, and stem cells. Representative useful stem cells include embryonic stem cells and non-embryonic stem cells; hematopoietic stem cells, bone marrow stem cells, neural stem cells, epithelial stem cells, skin stem cells, muscle stem cells, and adipose stem cells; as well as human stem cells, mouse stem cells, and rat stem cells. For cell growth and cell proliferation studies, cancer cells of all types can be added to the scaffolds.

In certain embodiments, the scaffold of the invention is a pristine porous chitosan scaffold. As used herein, the term “pristine” refers to a chitosan scaffold that includes only chitosan. It will be appreciated that pristine chitosan scaffolds of the invention may include trace (e.g., residual) amounts of components or materials used in the preparation of the scaffolds (e.g., acetate from the acetic acid solution from which the scaffold was formed). As the scaffolds are prepared from aqueous acidic chitosan solutions, the scaffolds of the invention may include trace (e.g., residual) amounts of the acid or its salts, water, or any impurities associated with the chitosan starting materials, the acid, or water. As described herein, trace amounts include amounts up to about 5 percent by weight based on the total weight of the scaffold. In certain embodiments, trace amounts include amounts up to about 2 percent by weight based on the total weight of the scaffold. In other embodiments, trace amounts include amounts up to about 1 percent by weight based on the total weight of the scaffold.

In certain embodiments, the methods of the invention produce pristine porous chitosan scaffolds. It will be appreciated that additional components may be added to the pristine porous chitosan scaffolds so produced (i.e., after scaffold formation) to provide porous scaffolds having a variety of uses. For example, in certain embodiments, cells are introduced into the pristine porous scaffolds for the purpose of culturing cells in the scaffold or for tissue engineering purposes.

Methods of Using the Porous Chitosan Scaffolds

In another aspect of the invention, methods for using the porous chitosan scaffolds are provided. The chitosan scaffolds of the invention can be used for any of the purposes known in the art for which porous chitosan or porous chitosan-containing scaffolds have been used. Representative uses of the chitosan scaffolds of the invention include those described in U.S. Pat. No. 7,736,669; U.S. Pat. No. 8,147,858; U.S. Pat. No. 8,460,692; U.S. Pat. No. 8,609,133; U.S. patent application Ser. No. 13/453,672 (US 2012/0272347); U.S. patent application Ser. No. 12/892,720 (US 2011/00762254); and PCT/US2013/035848 (WO 2013/155114), each expressly incorporated herein by reference in its entirety.

The invention provides a method for culturing cells or expanding a population of cells. In one embodiment, the method includes:

(a) seeding a porous scaffold of the invention with cells; and

(b) culturing the cells in the scaffold to provide a scaffold populated with the cells.

The invention provides a method for tissue engineering. In one embodiment, the method includes introducing a scaffold of the invention populated with cells into a tissue to be engineered.

For these methods, suitable cells and additives for cell growth for tissue engineering and cell culture uses are as described above.

Methods for Making the Porous Chitosan Scaffolds

In a further aspect, the invention provides methods for making porous chitosan scaffolds. The chitosan scaffolds of the invention are prepared from aqueous acid solutions of chitosan having high chitosan concentration (e.g., 2, 4, 8, and 12 percent by weight chitosan). The mechanical strength of the product scaffolds is due in part to the chitosan concentration in the aqueous acid solutions of chitosan from which the scaffolds are prepared. The higher the chitosan concentration in these solutions, the thicker the cell walls in the product scaffolds and the greater the mechanical strength of the scaffold.

In one embodiment, the invention provide a method for making a porous chitosan scaffold, comprising:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity, and wherein the second aqueous chitosan solution does not include an additional solvent (e.g., organic solvent) or additive (e.g., for increasing the strength of chitosan);

(c) freezing the second aqueous chitosan solution to provide a frozen chitosan composition; and

(d) freeze-drying the frozen chitosan composition to provide a porous chitosan scaffold.

In this embodiment, the aqueous chitosan solution that is frozen, and that ultimately provides the scaffold of the invention, does not include any additional materials, such as a solvent or strength additive. In the method of the invention, the process and materials provide the scaffolds having high mechanical strength without the addition of additional materials or process steps. Additional solvents that are not included in the method of the invention include solvents that do not dissolve chitosan, such as C3-C8 aliphatic alcohols having one hydroxy group, ethylene glycol monoethylether, ethylene glycol monobutylether, dioxane, THF, dimethylcarbonate, acetone, and acetonitrile (see US 2008/0242850).

In another embodiment, the invention provides a method for making a porous chitosan scaffold, consisting essentially of:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity;

(c) freezing the second aqueous chitosan solution to provide a frozen chitosan composition; and

(d) freeze-drying the frozen chitosan composition to provide a porous chitosan scaffold.

In a further embodiment, the invention provides a method for making a porous chitosan scaffold, consisting of:

(a) combining chitosan and an aqueous acid solution to provide a first aqueous chitosan solution having a first viscosity;

(b) conditioning the first aqueous chitosan solution to provide a second aqueous chitosan solution having a second viscosity, wherein the second viscosity is greater than the first viscosity;

(c) freezing the second aqueous chitosan solution to provide a frozen chitosan composition; and

(d) freeze-drying the frozen chitosan composition to provide a porous chitosan scaffold.

In certain embodiments, the above methods provide porous chitosan scaffolds having a compressive modulus from about 3 to about 20 MPa and compressive strength from about 0.2 to about 2.0 MPa.

In the above methods, the chitosan is dissolved in an aqueous acid solution. In certain embodiments, the acid is a weak acid. Suitable weak acids include organic acids (e.g., carboxylic acids). Representative weak acids include acetic acid, lactic acid, formic acid, and mixtures thereof. In other embodiments, the acid is a strong acid. Suitable strong acids include hydrochloric acid, sulfuric acid, nitric acid, phosphoric acid, and mixtures thereof.

In certain embodiments, the aqueous acid concentration is from about 0.5 to about 5 volume percent acid.

The scaffolds of the invention can be prepared from chitosan having a weight average molar mass from about 5,000 to about 500,000. In certain embodiments, the chitosan has a weight average molar mass from about 20,000 to about 200,000. Chitosan having a range of deacetylation is suitable for making the scaffolds. In certain embodiments, the chitosan is from about 60 to about 100% deacetylated. In other embodiments, the chitosan is from about 85 to about 95% deacetylated. In one embodiment, the chitosan has a Brookfield viscosity of from about 200 to about 800 cps in 1 weight percent in 1 percent acetic acid.

The methods of the invention provide for the use of relatively high chitosan concentrations. In general, the greater the chitosan concentration used in the method, the greater the mechanical strength of the product scaffold. In certain, the concentration of chitosan in the first aqueous chitosan solution is from about 2 to about 12 weight percent (e.g., 2, 4, 8, 12 weight percent) chitosan based on the total weight of the solution. The strength of the scaffold and therefore the chitosan concentration can be adapted depending on the ultimate use of the scaffold. For example, for hard tissue engineering, chitosan concentrations of about 8 weight percent are useful, and for soft tissue engineering, chitosan concentrations of about 2 weight percent are useful.

In the methods of the invention, the first aqueous chitosan solution is conditioned to maximize the dissolution, decrease protonation, and increase crystallinity of the chitosan. In the conditioning step, chitosan protonation is decreased resulting in an increase of chitosan crystallinity. In certain embodiments, conditioning includes maintaining the solution at a temperature from about 4 to about 30° C. (e.g., 25° C.) for 12 to 48 hours (e.g., 24 hours) with gentle stirring.

In the methods of the invention, conditioning the first aqueous chitosan solution having a first viscosity provides a second aqueous chitosan solution having a second viscosity, wherein the second viscosity is greater than the first viscosity. During the conditioning, the viscosity of the aqueous chitosan solution increases. In certain embodiments, the second viscosity is about 25% greater than the first viscosity. In other embodiments, the second viscosity is about 50% greater than the first viscosity. In further embodiments, the second viscosity is about 75% greater than the first viscosity. The degree of viscosity increase will depend on the chitosan (molar mass, deacetylation degree, concentration), acid (strong or weak, concentration), and reaction temperature and time. In certain embodiments, the second viscosity is the equilibrium or uniform viscosity. As used herein, the terms “equilibrium viscosity” or “uniform viscosity” refer to the viscosity ultimately obtained for an aqueous acid solution of chitosan at given chitosan and acid concentrations and at a given temperature.

Once the second chitosan solution is obtained, the solution is frozen. In certain embodiments, freezing the second aqueous chitosan solution comprises freezing at a temperature of from about −196 to about 0° C. (e.g., −20° C.) for 12 to 48 hours (e.g., 24 hours).

Once the second chitosan solution is frozen, the scaffold is provided by freeze-drying. In certain embodiments, freeze-drying the frozen chitosan composition comprises freeze drying under reduced pressure at a temperature of from about −90 to about −5° C. (e.g., −89° C.). As used herein, the term “reduced pressure” refers to pressure less than atmospheric pressure, typically achieved through the use of a vacuum pump.

The scaffolds of the invention can be prepared having a variety of predetermined shapes. The shape can be determined by casting the first aqueous chitosan solution into a vessel prior to freezing. The vessel determines the shape of the product scaffold and may be a membrane, block, tube, cylinder, bead, or sphere, among others.

In certain embodiments, the methods of the invention include removing air bubbles from the first aqueous chitosan solution prior to freezing. Removal of air bubbles can be achieved by a variety of means including centrifugation the solution or subjecting the solution to reduced pressure. Removal of air bubbles can result in the production of scaffolds having increased pore size uniformity and with increased number of open and interconnected pores.

In certain embodiments, the scaffolds of the invention and the methods for making these scaffolds are described as “comprising” the recited component and steps. In these embodiments, the scaffolds and methods may include components and steps in addition to those recited. In other embodiments, the scaffolds and methods are described as “consisting essentially of” the recited component and steps. In these embodiments, the scaffolds and methods may include components and steps in addition to those recited so long as the additional components and steps do not materially affect the characteristic properties (e.g., mechanical strength) of the scaffolds and methods of the invention. For example, the scaffolds of the invention do not include any non-recited component that would enhance the mechanical strength of the scaffold such as a reinforcement agent, a synthetic polymer, or another polymer that complexes to chitosan to enhance scaffold strength. Examples of components that do not materially affect the characteristic properties of the scaffold include components included in or added to scaffolds for their use, such as the inclusion or addition of components to facilitate, assist with, or enhance the growth of cells in and surrounding the scaffold. In further embodiments, the scaffolds and methods are described as “consisting of” the recited component and steps. In these embodiments, the scaffolds and methods do not include any components or steps other than those specifically recited.

The preparation, characterization, and use of representative chitosan scaffolds of the invention are described below.

Chitosan Solution Processing

The porous chitosan scaffolds were prepared by freeze-drying in combining with improved chitosan solution processing. Chitosan [poly(1,4-β-D-glucopyranosamine)], commonly produced by N-deacetylation of chitin, is soluble in acidic solvents. Acid solutions such as acetic, lactic, and formic acid are generally used for chitosan scaffold preparation. Chitosan in acidic solution becomes polyelectrolyte due to protonation, i.e., the proton (H⁺) in solution transforms —NH₂ groups into —NH₃⁺ groups following the equilibrium reaction:

Chitosan−NH₂+H₃O⁺ [symbol] Chitosan−NH₃⁺+H₂O

The positively charged —NH₃⁺ groups in adjacent polymer chains repel each other leading to chain expansion. Increasing solvent's acidity promotes —NH₃⁺ formation, facilitating chitosan dissolution, but excessive polymer chain expansion may lead to the loss of the initial chain arrangement, likely reducing polymer crystallinity upon solidification. As the crystallinity of a solid can be a determinant factor of material's mechanical properties. It is hypothesized that by using appropriate solvents, and adjusting acidity and polymer concentration, mechanical properties of resultant chitosan scaffolds can be controlled. Here, solutions with four chitosan concentrations (4, 6, 8 and 12 wt %) were prepared by dissolving chitosan in weak acetic acid solvent for a long time (a day versus a few minutes in common practice). A weak acid is less likely to cause complete protonation of —NH₂ groups. The long dissolution time facilitates the diffusion of acid molecules throughout chitosan and makes it possible to dissolve a high concentration of chitosan in a solvent of weak acid. In common practice, it is very difficult to dissolve chitosan at a concentration above 4 wt % in acetic acid because of the gelation of chitosan solution at high polymer concentrations. Viscosity profiles of chitosan solutions with varying dissolution times were measured to select optimal duration for chitosan dissolution.

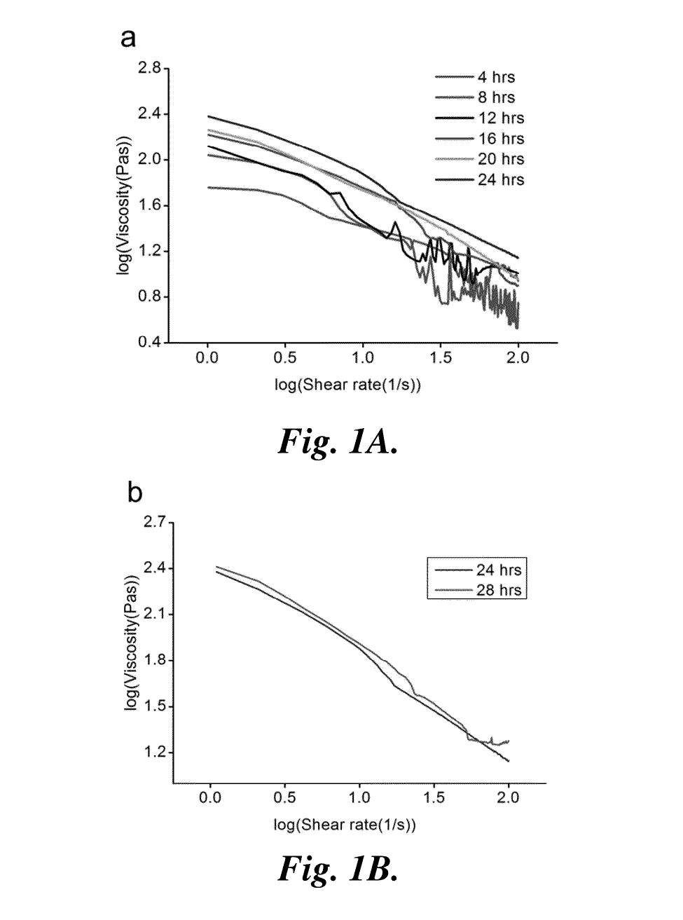

6 wt % bulk chitosan solutions were prepared and the viscosity was measured at different time points after mixing chitosan powder with solvent. 6 wt % chitosan solution was used due to its moderate viscosity. The solution viscosity increased over time (FIG. 1A), indicating that not all the chitosan molecules were readily dissolved. Rather, the dissolution of the polymer matrix occurs gradually in several steps: (1) diffusion of water and H⁺ ions to the nearest polymer chains of the bulk polymer matrix, (2) protonation of the polymer chains to form a gel layer, (3) disentanglement of polymer chains out of the gel layer to the polymer-solution interface, (4) further protonation of the polymer resulting in a greater —NH₃⁺ charge density that causes greater intra-molecular electrostatic repulsion among neighboring —NH₃⁺ groups, and (5) diffusion of the disentangled polymer chains into the bulk solution. As a result, chitosan molecule protonation increases over time, leading to more rigid chitosan chain conformation and thus higher viscosity of the solution.

However, after 24 hrs, no further changes in viscosity were observed (FIG. 1B). This indicates that an equilibrium was reached between the dissociation of acetic acid to H₃O⁺ ion and the protonation of —NH₂ groups to —NH₃⁺ groups within 24 hrs. A similar trend was observed for chitosan concentration up to 8 wt % (data now shown).

Morphology and Porosity of Chitosan Scaffolds

For tissue engineering, scaffolds should have a highly porous structure with interconnectivity that maintains a good nutrient flow and metabolic exchange for cell proliferation and tissue growth. The porous structure of our chitosan scaffolds was created by freezing chitosan solution to induce a phase separation and subsequent sublimation of solvent. SEM micrographs in FIGS. 2A-2D show the cross sections of chitosan scaffolds prepared from solutions with chitosan concentrations of (a) 4 wt %, (b) 6 wt %, (c) 8 wt %, and (d) 12 wt %. All chitosan scaffolds were highly porous, with pore sizes ranging from 100 to 500 μm, a size range suitable for bone tissue engineering. Scaffold porosity, measured by mercury intrusion porosimetry, decreased from 94.5±0.91%, 92.7±1.32%, 89.9±1.78%, to 86.1±2.13% as chitosan concentration increased from 4, 6, 8 to 12 wt %, while the pore wall thickness increased from 19±7 μm, 23±8 μm, 36±11 μm, to 45±14 μm, respectively. The thicker wall would result in higher mechanical strength as well as better structural integrity.

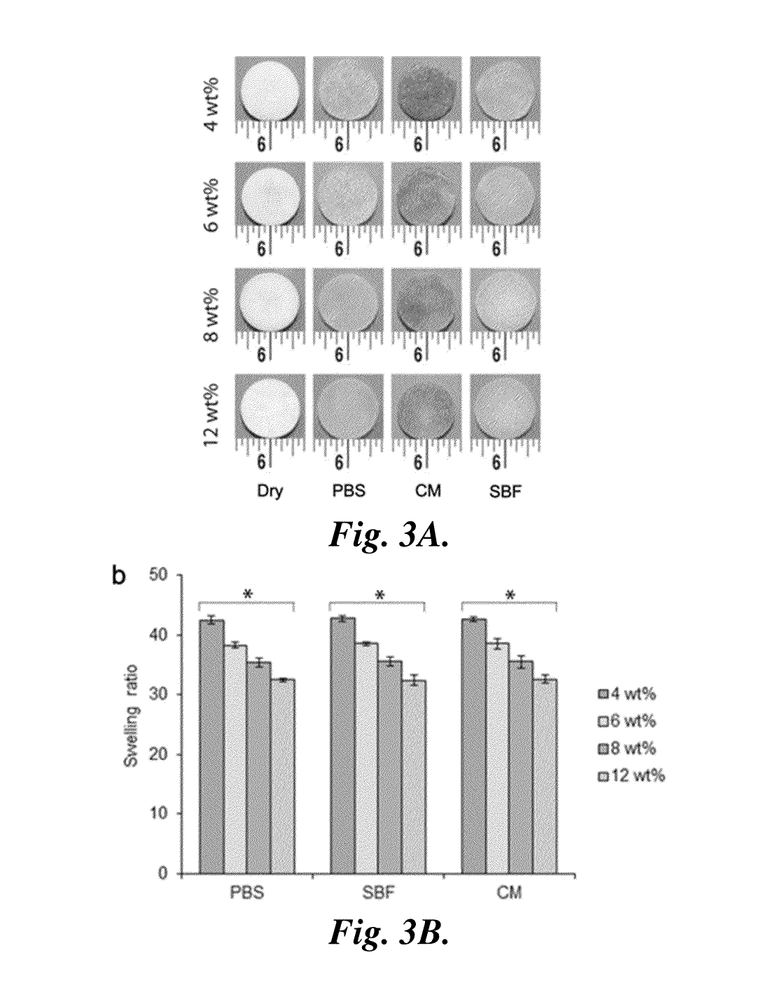

Swelling behavior and structural stability of scaffolds are critical for their practical use in tissue engineering. Most natural polymers, including chitosan, swell readily in biological fluids. In vitro culture studies indicated that initial swelling is desirable and the resultant increase in pore size facilitates cell attachment and growth. However, continuous swelling would lead to the loss of mechanical strength and introducing additional compressive stress to surrounding tissue. To assess the swelling behavior of the produced scaffolds, the scaffold samples were soaked in DMEM culture media, PBS, and simulated body fluid (SBF) for up to two weeks. No apparent size/shape change was observed after during the two-week period for all the scaffolds in either of these media (FIG. 3A). The swelling was also accessed quantitatively by evaluation of the swelling ratio which is defined as the weight difference between wet and dry states of a sample divided by the dry weight of the sample. This ratio reveals how the material absorbs aqueous media with respect to its dry state. FIG. 3B shows the equilibrium swelling ratios of chitosan scaffold samples produced from 4, 6, 8, and 12 wt % chitosan solutions after two weeks of sample immersion in the media. It can be seen that scaffolds with higher chitosan concentrations have smaller swelling ratios, which validates the earlier speculation that greater wall thickness helps retain the structural integrity of the scaffold in wet condition. Moreover, regardless of the medium used, swelling ratios were about the same for scaffolds with a same chitosan concentration. Swelling ratios were also measured at 2, 7, and 14 days and no changes were observed over time. The swelling ratios were assessed with finer time intervals and found the equilibrium levels were reached in 1 hour.

Mechanical Properties of Chitosan Scaffolds

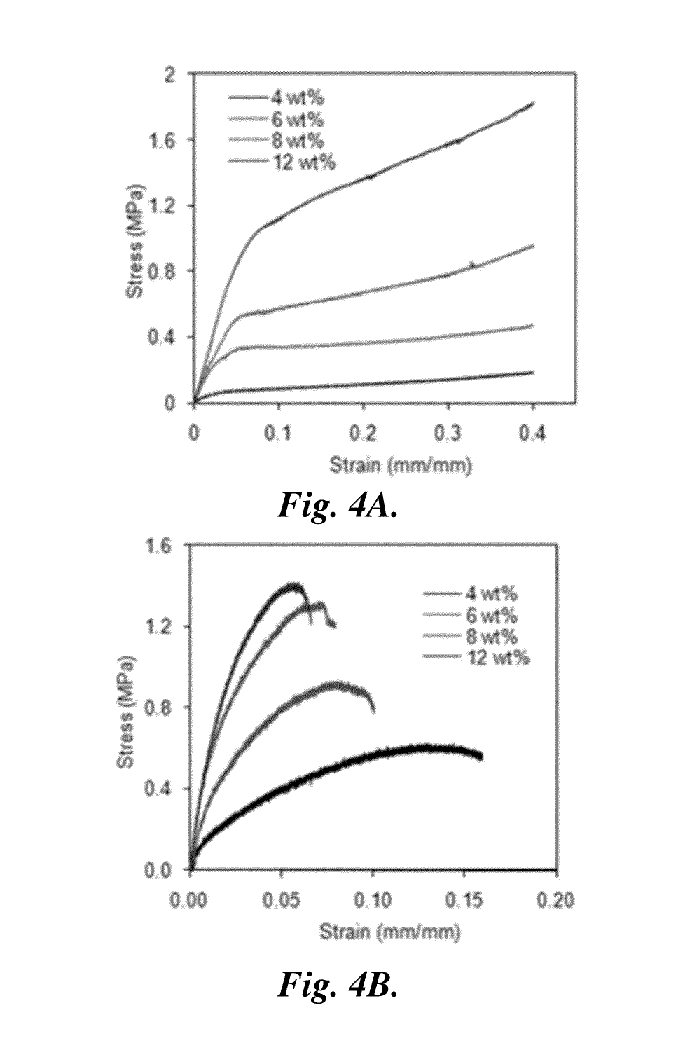

For bone tissue engineering, scaffolds should have sufficient mechanical strength for bone regeneration at the site of implementation and maintain structural integrity during both in vitro and in vivo cell growth. Compression tests were performed on each type of chitosan scaffolds (n=5 per condition) to obtain the stress-strain relation from which modulus and strength were evaluated. Mechanical properties of the scaffolds improved markedly with increasing chitosan concentration (FIG. 4). Compressive strength and modulus increased from 0.31±0.02 MPa and 5.56±0.38 MPa (4 wt %), respectively, to 1.74±0.01 MPa and 17.99±0.11 MPa (12 wt %); a more than 5-fold increase in strength and a more than 3-fold increase in modulus (see Table 1). Similarly, tensile strength and modulus also increased from 0.58±0.02 MPa and 22.86±1.20 MPa (4 wt %), respectively, to 1.38±0.02 MPa and 67.38±1.28 MPa (12 wt %) (see Table 1).

The mechanical properties of representative chitosan scaffold depicted in FIGS. 4A and 4B. Stress-strain relations of chitosan scaffolds prepared at different chitosan concentrations and acquired in (a) compression tests and (b) tensile tests.

The compressive and tensile strength and moduli of representative chitosan scaffolds of the invention are shown in Table 1.

TABLE 1
Summary of mechanical properties of chitosan scaffolds prepared
from solutions of different chitosan concentrations (wt %).
Data refer to mean value ± standard deviation.
Compression tests

| Chitosan | | Strength (MPa) |
|---|---|---|
| scaffold specimens | Modulus (MPa)* | (at 40% strain)* |
| 4 wt % | 5.56 ± 0.38 | 0.31 ± 0.02 |
| 6 wt % | 10.08 ± 0.87 | 0.50 ± 0.01 |
| 8 wt % | 15.77 ± 0.98 | 0.96 ± 0.04 |
| 12 wt % | 17.99 ± 0.51 | 1.74 ± 0.08 |

Tensile tests

| | Modulus (MPa)* | Strength (MPa)* |
|---|---|---|
| 4 wt % | 22.86 ± 1.20 | 0.58 ± 0.02 |
| 6 wt % | 40.27 ± 0.84 | 0.97 ± 0.09 |
| 8 wt % | 62.63 ± 1.19 | 1.28 ± 0.04 |
| 12 wt % | 67.38 ± 1.28 | 1.38 ± 0.02 |

*p < 0.05, by Student's t-test, n = 5, all values in each mechanical property category were found to be significantly different from each other.

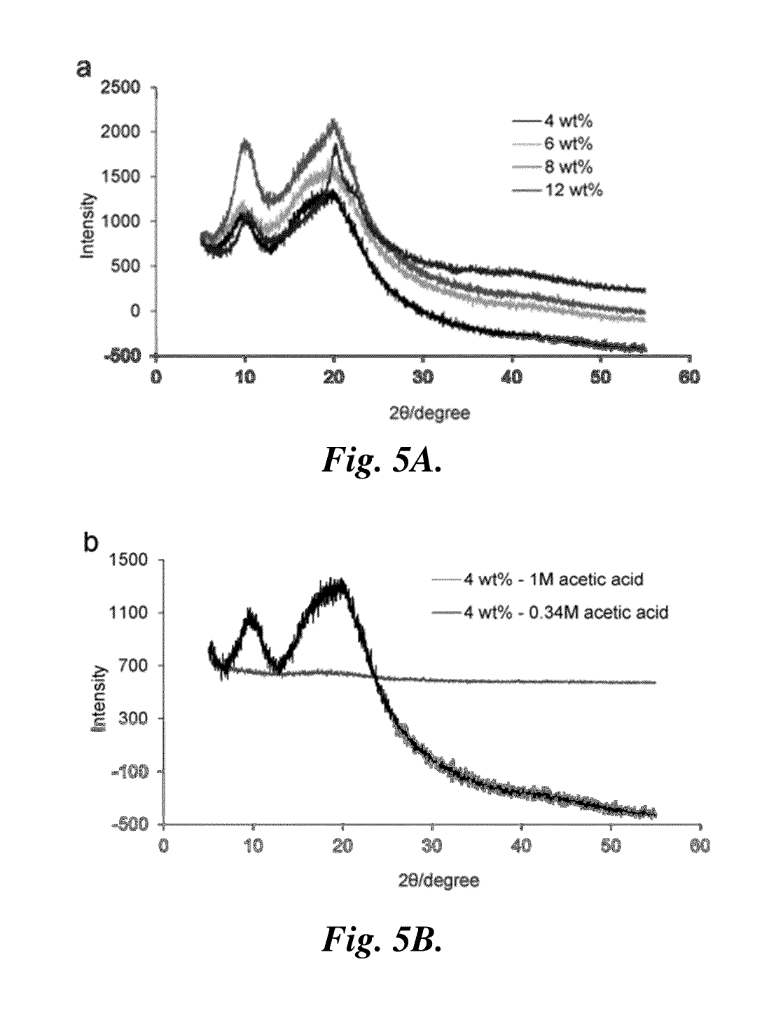

This enhancement in mechanical strength of the scaffold with increasing chitosan concentration or decreasing relative acid concentration (i.e., acid amount per unit chitosan) can be attributed to the increase in chitosan mass per unit volume as well as the increased crystallinity. The former is evident by microstructural changes of the scaffold, where the pore wall thickness increased and porosity decreased with increasing chitosan concentration (FIGS. 2A-2D). To illustrate the role of chitosan crystallinity in regulating the mechanical property, X-ray diffraction (XRD) patterns were acquired from chitosan films prepared from chitosan solutions used for fabricating the scaffolds (FIG. 5A). Two characteristic peaks around 10° and 20° in the XRD patterns are attributed to crystalline molecular alignment. Intensities of these characteristic peaks increased with increasing chitosan concentration, indicating the higher degree of crystallinity exhibited by the scaffolds of higher chitosan concentrations and mechanical strength. However, this increase in crystallinity was markedly lessened as the chitosan concentration increased to above 8 wt %. This phenomenon can be explained in terms of degree of protonation.

When chitosan is dispersed in acetic acid solution, following equilibrium conditions are observed:

CH₃COOH+H₂O [symbol] CH₃COO⁻+H₃O⁺

Chit-NH₂+H₃O⁺ [symbol] Chit-NH₃⁺+H₂O

The concentration of H₃O⁺ in solution is dependent on acetic acid concentration and protonation of chitosan. The pH value of the chitosan solution, which is essentially a measure of the effective concentration of H₃O⁺, increased over time. The pH values of 4, 6, 8, and 12 wt % chitosan solutions were 3.88, 3.93, 3.96, and 3.98 respectively at initial dissolution, and increased to 4.22, 4.48, and 4.87 at 12 hrs, and to 4.42, 4.71, and 5.68 at 24 hrs, respectively. The corresponding degrees of protonation determined from these pH values were 0.585, 0.508, and 0.416, respectively. This means that more chitosan chains were intact in the solutions with higher chitosan concentrations and thus higher degrees of crystallinity were obtained upon solidification of these solutions.

For 12 wt % solutions, the pH value was not found to be constant throughout the solution; it varied between 4.62-5.12 at 12 hrs and between 5.34-6.26 at 24 hrs. This is likely because there is insufficient amount of solvent to completely disperse H₃O⁺, and the protonation of chitosan occurred non-uniformly in the solution. Thus, the degree of crystallinity only moderately changed when the chitosan concentration increased from 8 to 12 wt %. Interestingly, this correlates well with the changes in mechanical properties (see Table 1), which shows that when the chitosan concentration increased from 8 to 12 wt %, only a slight increase in mechanical strength and modulus was observed, considerably less than the increases when chitosan concentration increased for the same concentration span from 4 to 8 wt %. This may imply that the slight increase in strength and modulus as chitosan concentration increased from 8 to 12 wt % was dominantly attributed to the increase in polymer mass per unit volume.

To further illustrate that the acid concentration is a key factor regulating chitosan crystallinity and thus the mechanical properties, chitosan scaffolds and films were prepared from two solutions with the same chitosan concentration (4 wt %) but different acetic acid concentrations (0.34 M and 1 M). This precludes the contribution of chitosan mass difference to the structural and mechanical properties. XRD analysis indicated that 1 M solution films had almost an amorphous structure, while 0.34 M solution films demonstrated an apparent degree of crystallinity (FIG. 5B). Compressive strength and modulus of 1 M solution scaffolds were also significantly lower than those of 0.34 M solution scaffolds (0.08±0.005 MPa and 1.334±0.03 MPa versus 0.31±0.02 MPa and 5.56±0.38 MPa, respectively). Because both scaffolds were prepared at the same chitosan concentration, these results confirmed the strong influence of solvent acidity on chitosan crystallinity and thus on scaffold mechanical properties.

Osteoblast-Like MG-63 Cell Attachment and Proliferation

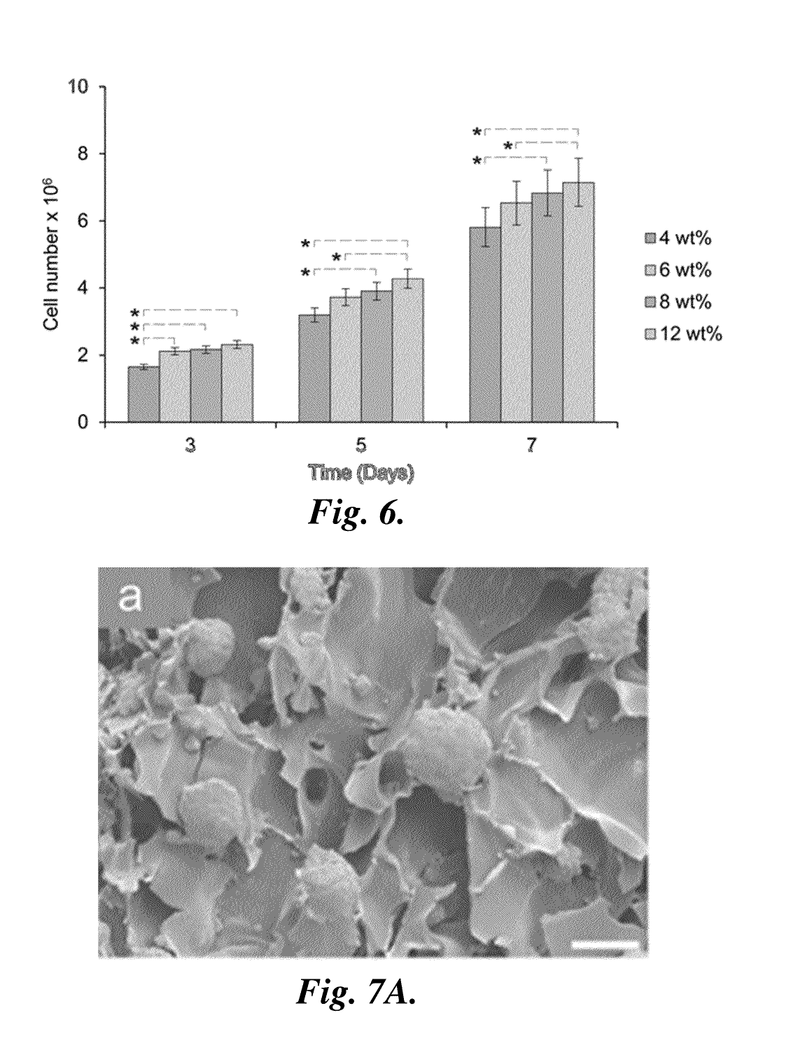

Studies have shown that cell adhesion and proliferation are regulated by physical, chemical, and mechanical cues of the biomaterial. It is known that chitosan provides chemical cues favorable for cell attachment and proliferation, but little is known about how chitosan concentration or scaffold mechanical properties affect the cell-material interaction, largely because of the challenge in making chitosan scaffolds of varying mechanical strengths. Osteoblast-like MG-63 cells, a model hard tissue cells, were seeded onto chitosan scaffolds of different mechanical properties and cultured for 7 days in standard culture media (Dulbecco's modified eagle medium (DMEM) with 10% FBS and 1% antibiotic-antimycotic) without osteogenic reagents. MG-63 cells express a number of characteristic features of osteoblasts that are responsible for bone formation. Cells were seen to proliferate well on all scaffolds, and scaffolds with higher mechanical strength better supported cell proliferation (FIG. 6). These results indicate that mechanical properties of the chitosan scaffold play a role in regulating cell growth, with scaffolds of higher mechanical strength more effective in supporting cell proliferation.

Osteoblast-Like MG-63 Cell Morphology and Function

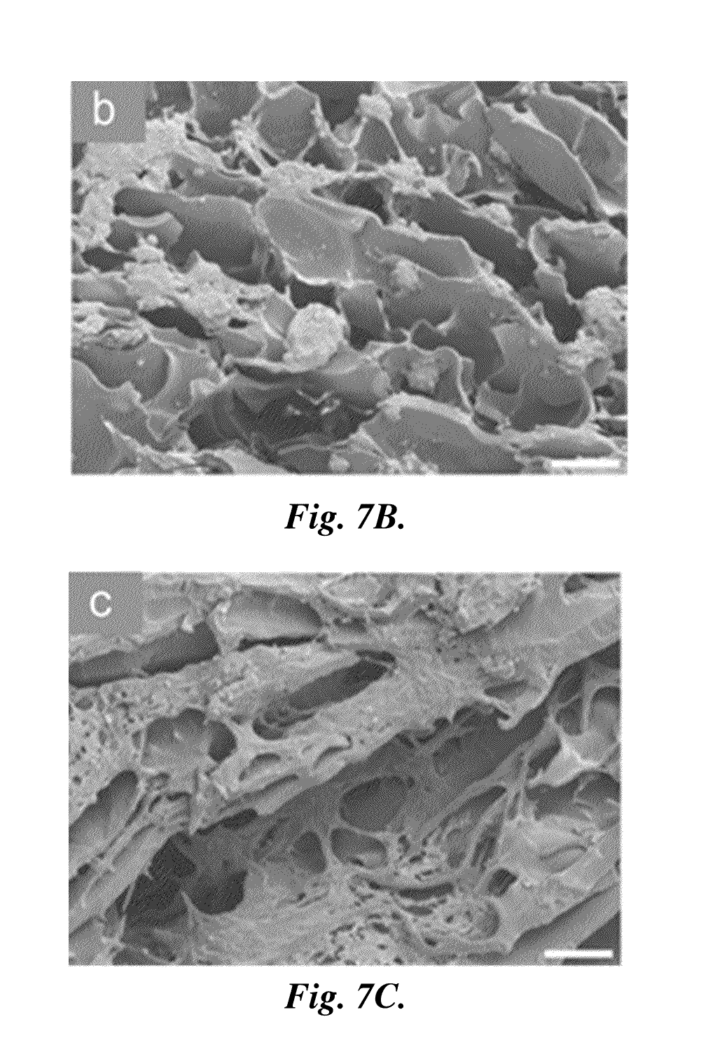

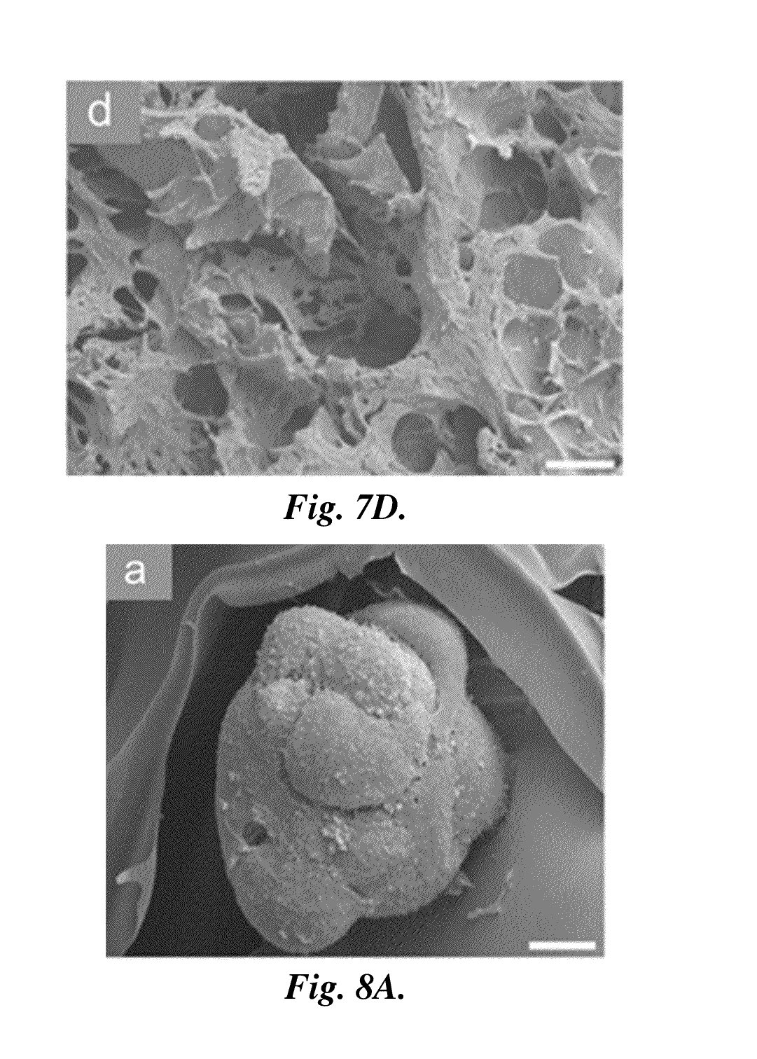

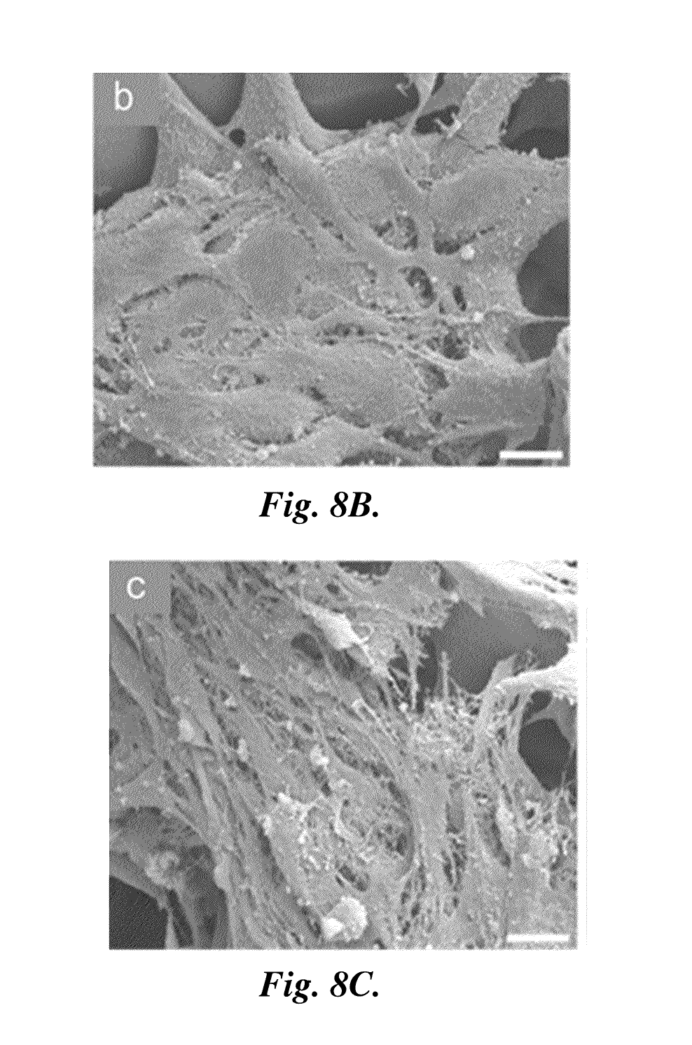

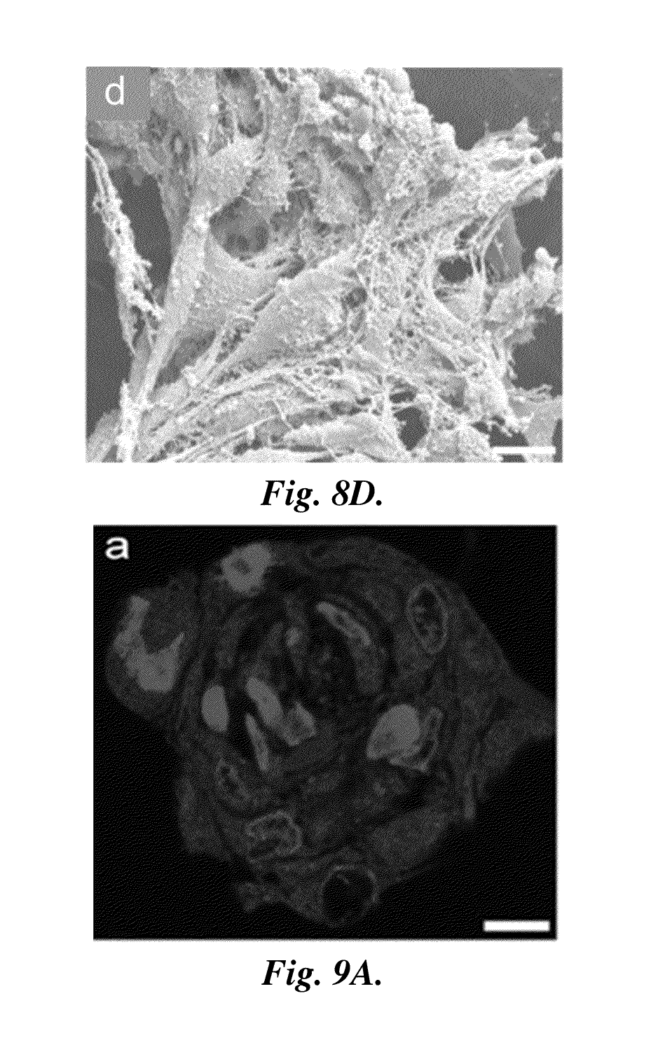

SEM imaging of cell-cultured scaffolds showed that MG-63 cells grown on scaffolds of lower chitosan concentrations displayed a round shape and formed aggregates after 7 days of culture, while those grown on scaffolds of higher chitosan concentrations exhibited an elongated shape and spread out discretely within the pores and on the surface of the scaffold (FIGS. 7A-7B), signifying better cell adhesion and osteogenic activity. SEM micrographs at higher magnification (FIGS. 8A-8B) show that the surface of the cells on the 4 and 6 wt % chitosan scaffolds appeared to be smoother than that on the 8 and 12 wt % chitosan scaffolds, indicating greater extracellular matrix (ECM) depositions and fibrous networks on 8 wt % and 12 wt % scaffolds than on 4 wt % and 6 wt % scaffolds. This suggests that chitosan scaffolds of higher mechanical strength better support osteogenic activity.

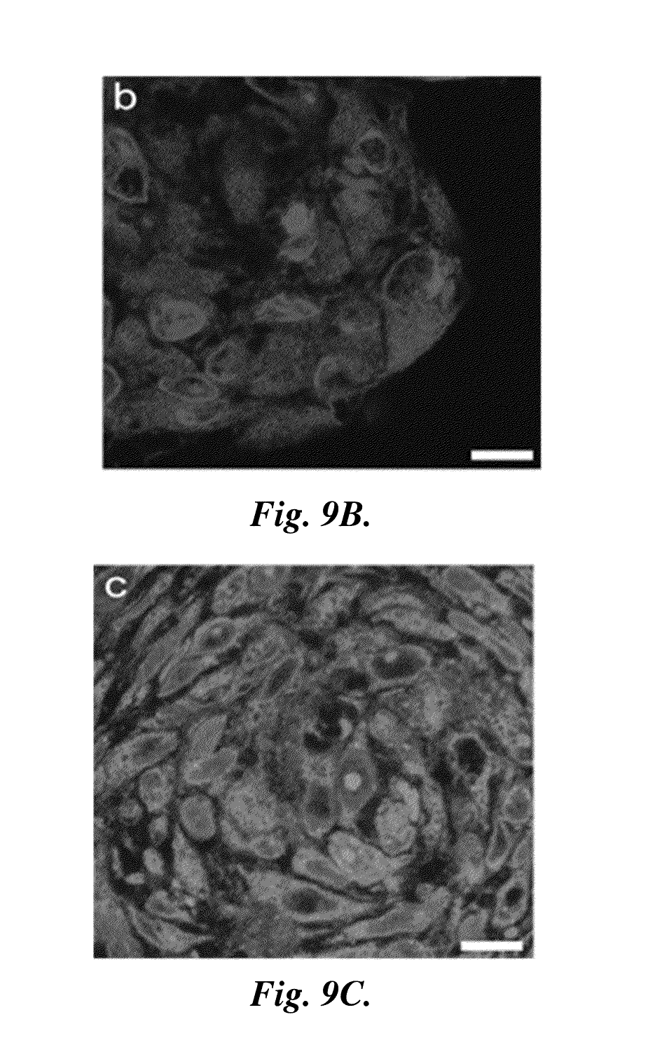

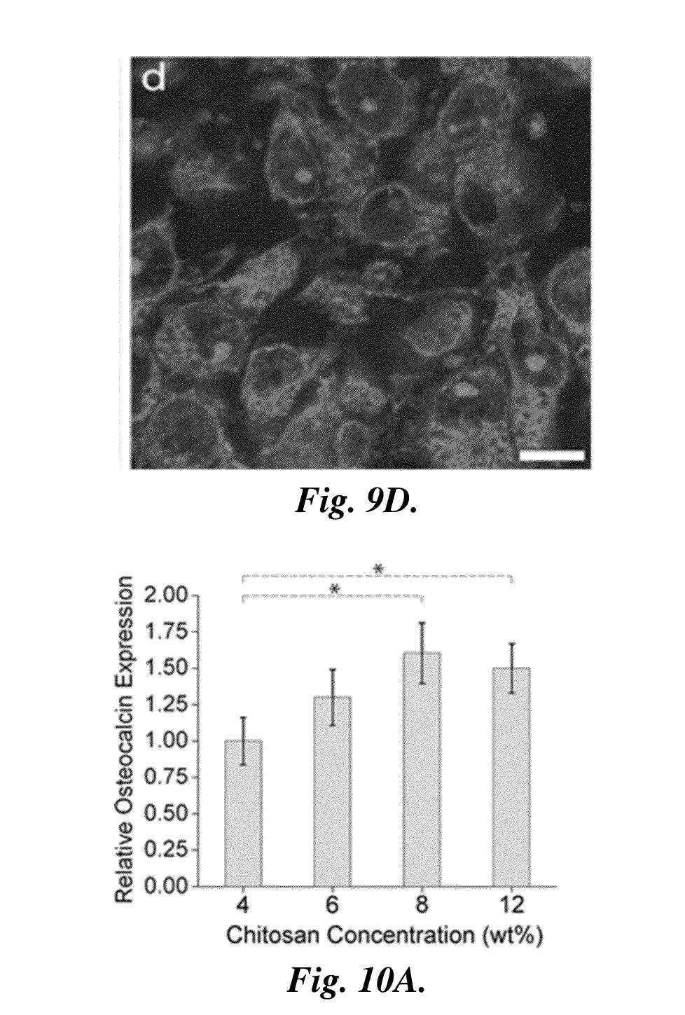

Cells demonstrate mechanotransductive responses by probing the stiffness of their substrate, among other external mechanical signals, and reacting via biochemical mediators such as the cytoskeleton. Thus, the choice of scaffold depends on the specific tissue to be engineered. It is generally recognized that mechanical properties of materials can influence the cellular behavior of osteoblasts. Studies have shown that stiffer hydrogels induce much higher alkaline phosphatase expression and mineral deposits by seeded cells than less stiffer hydrogels. To investigate the relationship between mechanical stiffness and functionality of osteoblast, osteogenic activity of the scaffolds with varying mechanical properties was further evaluated by osteocalcin immunostaining with osteocalcin primary antibody, followed by FITC conjugated IgG secondary antibody. Osteocalcin is a primary non-collagenous protein produced by osteoblasts, which signals terminal osteogenic differentiation, and is commonly used to measure new bone formation. Greater osteocalcin (green) deposits were seen in scaffolds of higher chitosan concentrations (high stiffness) (FIGS. 9A-9D).

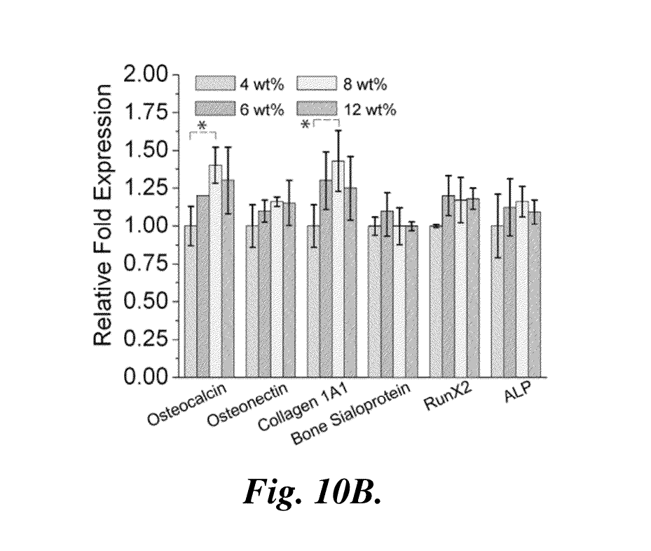

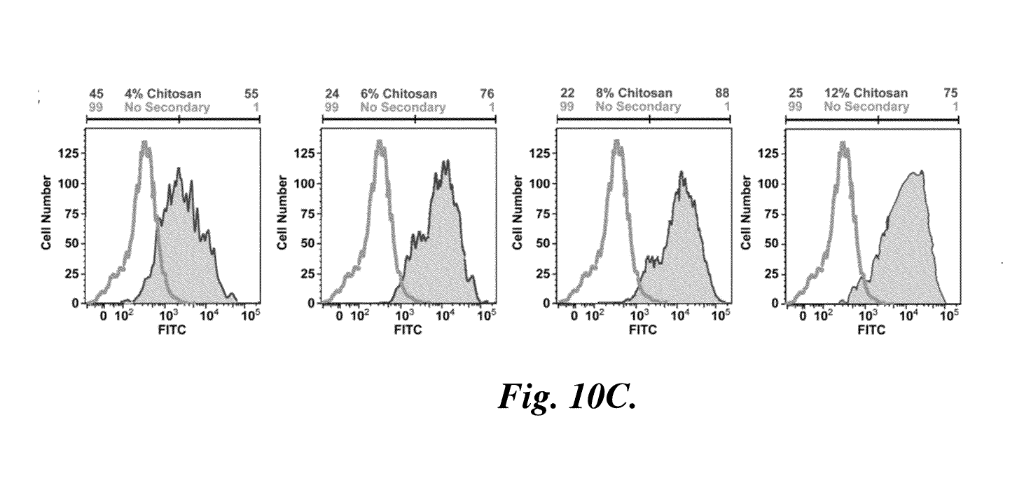

Osteocalcin deposits were further quantified by immunoblotting (FIG. 10A) and flow cytometry (FIG. 10C). Immunoblotting evaluates the total osteocalcin expression of MG-63 cells. Osteocalcin expression in MG-63 cells was seen to increase with chitosan concentration up to 8 wt % where osteocalcin expression was about 150% of that produced on 4 wt % chitosan scaffolds (FIG. 10A). This agrees well with the osteocalcin expression observed by confocal microscopy, confirming that the mechanical properties of the chitosan scaffold affect osteogenic response.

Osteocalcin, osteonectin, collagen1A1, bone sialoprotein, runx2, and alkaline phosphatase (ALP) are the most important genes expressed by osteoblasts that collectively take part in formation of the osseous matrix and controlled calcification. The expression patterns of these genes were quantified by RT-PCR using the primer sequences listed in Table 2. The expressions of all these genes except bone sialoprotein and runx2 mRNA were increased with the increase of chitosan concentration in scaffolds up to 8 wt % (FIG. 10B). The elevated expression of bone sialoprotein is limited to mature, mineralizing osteoblasts, which is expected only for cells cultured in media containing mineralization agent. The expression of runx2 is only elevated during early differentiation to osteogenic lineage, and maintained at steady levels after initial commitment. Thus, the result suggests that MG-63 in all scaffolds were well committed to the osteogenic lineage.

TABLE 2
Primer sequences for RT-PCR analysis of MG-63 cell culture

| Primers | Forward Sequence | Reverse Sequence |
|---|---|---|
| osteocalcin | 5-AAAGCCCAGCGACTCT-3 | 5-CTAAACGGTGGTGCCATAGAT-3 |
| | SEQ ID NO: 1 | SEQ ID NO: 2 |
| osteonectin | 5-ACAAGCTCCACCTGGACTACA-3 | 5-TCTTCTTCACACGCAGTTT-3 |
| | SEQ ID NO: 3 | SEQ ID NO: 4 |
| Collagen 1A1 | 5-TCCTGCCGATGTCGCTATC-3 | 5-CAAGTTCCGGTGTGACTCGTG-3 |
| | SEQ ID NO: 5 | SEQ ID NO: 6 |
| Bone | 5-CAGAGGAGGCAAGCGTCACT-3 | 5-CTGTCTGGGTGCCAACACTG-3 |
| sialoprotein | SEQ ID NO: 7 | SEQ ID NO: 8 |
| runX2 | 5-GCTTCTCCAACCCACGAATG-3 | 5-GAACTGATAGGACGCTGACGA-3 |
| | SEQ ID NO: 9 | SEQ ID NO: 10 |
| ALP | 5-TCGCCTACCAGCTCATGCATAACA-3 | 5-TGAAGCTCTTCCAGGTGTCAACGA-3 |
| | SEQ ID NO: 11 | SEQ ID NO: 12 |
| Beta-actin | 5-CAGGATTCCATACCCAAGAAG-3 | 5-AACCCTAAGGCCAACCGTG-3 |
| | SEQ ID NO: 13 | SEQ ID NO: 14 |

The osteocalcin expression profile obtained from flow cytometry showed that osteocalcin expression levels were 55, 76, 88, and 75% in 4, 6, 8, and 12 wt % chitosan scaffolds, respectively (FIG. 10C), which confirms that scaffolds of higher chitosan concentrations (up to 8 wt %) were more effective in supporting osteoblast cell maturation. This result corroborates the confocal and immunoblotting data. All three quantitative assays of osteogenic activity agree with the EM and confocal images shown in FIGS. 9A-9D and confirm that chitosan scaffolds with higher mechanical strength were more conducive for bone tissue engineering.

In summary, in certain embodiments, the present invention provides porous pristine chitosan scaffolds with unprecedented high mechanical strength. The compressive modulus and strength of the scaffolds with high chitosan concentration were as high as 17.9 MPa and 1.74 MPa, respectively, more than 5-fold and 3-fold higher than those of scaffolds with the lowest chitosan concentration. Moreover, the integrity of scaffolds was intact in different bio-media, which is essential to their practical use in tissue engineering. It has also been demonstrated that increasing chitosan concentration also promotes the proliferation and osteogenic activity of a model hard tissue cell line like MG-63 cells. The results indicate that the chitosan scaffolds, with improved mechanical properties, would broaden the use of chitosan in tissue engineering and other biomedical applications.

The following examples are provided for the purpose of illustrating, not limiting, the invention

### EXAMPLES

### Example 1

### The Preparation of Representative Chitosan Scaffolds

In this example, the preparation of representative chitosan scaffolds of the invention is described.

Chitosan solutions of different concentrations (4, 6, 8 and 12 wt %) were prepared by dissolving chitosan powder (Medium molecular weight, weight average molar mass MW 190-200K, 85% deacetylated, Brookfield viscosity 200-800 cps in 1% solution with 1% acetic acid, Sigma-Aldrich) in 0.34 M (2 v %) acetic acid and maintaining the solutions at 25° C. for 24 h with intermittent stirring. Chitosan was dissolved completely in 4, 6 and 8 wt % solutions. The solution without filtration was then cast into a 10 mL plastic syringe, and centrifuged at 6000 rpm for 10 min to remove air bubbles. The samples were then frozen at −20° C. for 24 h. After freezing, the samples were lyophilized in a freeze drier (Labconco FreeZone 6Plus) under vacuum at −89° C. until fully dried. The resultant cylindrical chitosan scaffolds (about 10 cm height and 15 mm diameter) were cut into specimens of different dimensions depending on experiment: rectangles (15 mm×10 mm×2 mm) for tensile tests, cylinders (9 mm thickness and 15 mm diameter) for compression tests, and discs (3 mm thickness and 15 mm diameter) for in vitro studies and SEM imaging. Prior to experiments, all specimens were immersed in 1 M sodium carbonate solution for 5 h to neutralize residual acetate functional groups, washed with DI water 3 times, immersed in DI water overnight to remove residual sodium salt, and then lyophilized.

### Example 2

### Methods for Characterizing Representative Chitosan Scaffolds

In this example, methods for characterizing representative chitosan scaffolds of the invention are described.

Chitosan Viscosity Measurements.

6 wt % chitosan solution was prepared by dissolving chitosan powder in 0.34 M acetic acid at room temperature with stirring. 10-mL samples were removed from the solution every four hours up to 28 hr. A Haake Viscotester VT550 Rheometer (HAAKE) with an SV sensor system was used to measure the viscosity of the sample. The sample was run at 25° C. maintained by a circulating water bath (DC-10, HAAKE), and measured for 1000 s up to a shear rate of 100 s⁻¹.

SEM Imaging.

Chitosan scaffold samples were sputter-coated with Au/Pd for 60 s at 18 mA and imaged by scanning electron microscopy (SEM, JEOL JSM 7000). For SEM analysis of the morphology of cells grown in chitosan scaffolds, the scaffold samples cultured with MG-63 cells for 7 days were rinsed in PBS, fixed in Karnovsky's fixative overnight, rinsed in DI water, and dehydrated by sequential incubations in 50, 75 and 100% ethanol for 15 min each at room temperature. The samples were then dried with a critical point dryer, sputter-coated with Au/Pd for 60 s at 18 mA and imaged by SEM. Wall thickness of the scaffolds was measured from SEM images (n=20).

XRD Analysis.

For XRD analysis, chitosan films were prepared by spin-coating chitosan solutions described above on Petri dishes, followed by freezing at −20° C. for 24 h and lyophilizing. The film specimens were treated in the same manner to remove residual acetate functional groups as described in scaffold preparation section, before XRD analysis (Bruker D8). By controlling the speed of the spin coater, the thickness of the films was kept closer to each other to avoid influence of film thickness on their crystallinity measurements. The thickness was measured at least at three places on a film by a screw gauge micrometer (Mitutoyo).

Porosity Measurement.

Scaffold porosity was measured by mercury intrusion porosimetry (AutoPoreIV 9500, Micromeritics). The Washburn equation was used to calculate the pore diameter. Porosity (%), total pore volume (ml/g), total pore area (m²/g), and pore size distribution of the scaffold were determined by measuring the volume of the mercury infused. For each measurement, cylindrical scaffolds of 3 mm in diameter and 3 mm in length were placed in a 10-mL penetrometer, subjected to a vacuum of 50 mm Hg, and infused with mercury. Samples were weighed before and after the mercury infusion.

Shape Retention Assessment.

Scaffold specimens of 3 mm thickness and 15 mm diameter were neutralized in 1M sodium carbonate solution for five hrs and rinsed with copious amount of water. The soaked scaffolds were frozen at −20° C. and freeze-dried. Dried samples were incubated at room temperature for two weeks in one of the following media: culture media (Dulbecco's modified eagle medium (DMEM), 10 v % FBS and 1 v % antibiotic/antimycotic), phosphate buffered saline (PBS, pH 7.4), and simulated body fluid (SBF, pH 7.25). Swelling of chitosan scaffolds in culture media, PBS and SBF were quantified by the swelling ratio defined by the equation:

G=(W_{wet}−W_{dry})W_{dry}

where G is the swelling ratio, and W_{wet} and W_{dry} are the weights of the scaffold in wet and dry conditions, respectively. The values were expressed in mean±standard deviation (n=3).

Mechanical Tests.

Compression and tensile tests (n=5 per condition) were performed using an Instron 5900 with a crosshead speed of 1 mm/min and 0.5 mm/min, respectively. The dry specimens were cylinders of 15 mm in diameter and 3 mm in thickness. To prevent buckling, specimens were compressed to about 40% of their original thickness. Tensile tests proceeded until specimen fractured.

Mechanical Tests.

Compressive strength (i.e., compressive yield strength) and compressive modulus of scaffolds (n=5 per condition) were performed using an Instron 5900 with 10 Kilo Newton load cells following the guideline in ASTM D5024-95a (Whang, K., et al., Tissue Eng. 5:35-51, 1999, expressly incorporated herein by reference in its entirety). The crosshead speed of the Instron tester was set at 1 mm/min and load was applied until the specimens were compressed to approximately 40% of their original thickness. The dry specimens were cylinders of 15 mm in diameter and 3 mm in thickness. To prevent buckling, specimens were compressed to about 40% of their original thickness. Tensile tests proceeded until specimen fractured.

In Vitro Studies.

Scaffolds were sterilized by soaking in 70% ethanol overnight, and then in culture media for 24 hrs before cell seeding. Specimens were each seeded with 1×10⁶ MG-63 osteoblast cells (ATCC) in 100 μL of standard culture media, DMEM with 10% FBS and 1% antibiotic-antimycotic, and incubated at 37° C. for 8 hrs before adding 1 mL standard culture media to each well. Cell proliferation was assessed for a 7-day period using the Alamar Blue assay (Invitrogen). Cell morphology in scaffolds was examined with SEM. Osteocalcin protein production was assessed by immunofluorescent staining with mouse anti-human osteocalcin primary antibody (Abcam) as primary antibody and FITC conjugated goat anti-mouse IgG antibody (Abcam) as secondary antibody, and DAPI staining for nuclei. Osteocalcin deposits was quantified by immunoblotting, real-time PCR (RT-PCR) and flow cytometry analyses.

Cell Proliferation.

The cell proliferation was assessed with the Alamar Blue assay (Invitrogen) at 3, 5 and 7 days after culture (n=3 for each time point for each condition). Samples were washed in PBS twice and incubated for 4 hrs at 37° C. in a 10% AlamarBlue reagent solution in DMEM phenol-free media (Invitrogen). The AlamarBlue reagent for each sample was transferred to a 96 well plate and read at A₅₇₀ and A₆₀₀ with a microplate reader (Molecular Devices) to calculate the percent reduction of the AlamarBlue reagent. The percent reduction of Alamar blue was determined following the manufacturer's protocol.

Immunostaining.

The osteocalcin protein production by MG-63 cells was assayed by immunofluorescent staining after one-week cell culture. The samples were fixed in 4% methanol-free paraformaldehyde (Aldrich) in PBS overnight at 4° C., washed in PBS, rinsed with DI water, and dehydrated by sequential incubations in 50, 75 and 100% ethanol for 15 min each at room temperature. The samples were then paraffinized to obtain 8 μm sections of scaffolds. The sections were deparaffinized in xylene and hydrated by sequential incubation in 100, 75, 50, 30% ethanol for 3 min each and then placed in cold running water for 10 min. The samples were then washed in ice-cold PBS, extracted in 0.25% (v/v) Triton X-100 (Sigma) in PBS for 30 min, rinsed with PBS, pre-incubated in 10% goat serum (Abcam, Cambridge, Mass.) in PBS for 30 min, and incubated overnight at 4° C. in mouse anti-human osteocalcin primary antibody at a 1:500 dilution in PBS with 0.25% Triton X-100. The samples were rinsed, incubated in FITC conjugated goat anti-mouse IgG antibody (Abcam) as secondary antibody at a 1:500 dilution in PBS for 2 hrs, rinsed with PBS, mounted to a coverslip, and stained with Prolong Gold Antifade reagent with DAPI (Invitrogen). The samples were cured overnight at room temperature and imaged with a confocal fluorescent microscope (Zeiss Meta Confocal, Germany).

Immunoblotting.

1×10⁶ MG-63 cells were cultured on each of chitosan scaffold samples for 7 days. The cells were detached from the sample with Versene (Gibco) at room temperature for 10 min, counted, frozen at −80° C., and lysed with RIPA buffer (Sigma). Cell lysate equivalent to 100,000 cells was resuspended in Laemmli buffer (BioRad), and spotted onto a PVDF membrane (BioRad). The membrane was probed with monoclonal mouse anti-human osteocalcin antibody (Abcam), labeled with alkaline phosphatase conjugated goat anti-mouse secondary antibody (BioRad) at 10 μg/ml, and visualized with Immun-Star alkaline phosphatase reagent (BioRad) on a ChemiDoc (BioRad). The relative intensity was measured with ImageJ.

Flow Cytometry.

1×10⁶ MG-63 cells were cultured on each of chitosan scaffold samples for 7 days. The cells were detached from the scaffold with Versene (Gibco) at room temperature for 10 min and processed for FACS analysis to detect osteocalcin positive cells. Monoclonal mouse anti-human osteocalcin antibody (Abcam) and FITC conjugated rabbit anti-mouse IgG secondary antibody (Abcam) were used at 10 μg/mL in a 3% suspension of BSA (Sigma) in PBS. Cells were analyzed with a BD FACSCanto flow cytometer (Becton Dickinson Biosciences).

Real-Time PCR (RT-PCR).

Cell-scaffold constructs were homogenized by vortexing and passing through QIAshredder columns. Total RNAs were isolated from MG-63 cells in scaffolds in triplicate using RNeasy, and 30 ng of total RNA for each sample was converted to cDNA using the QuantiTect Reverse Transcription Kit following the manufacturer's instructions (Qiagen). SYBR Green PCR Master mix (Qiagen) was used for template amplification with a primer for each of the transcripts examined. Thermocycling for all targets were carried out in a 30 mL solution containing 0.3 mM primers (Integrated DNA Technologies) and 4 pg cDNA from the reverse transcription reaction under following conditions: 15 s at 94° C., 30 s at 55° C., and 30 s at 72° C. over 40 cycles. The reaction was performed and analyzed in a CFX96 (BioRad).

Statistical Analysis.

Statistical analyses for the mechanical testing and gene expression were performed using one-way analysis of variance (ANOVA). p Values less than 0.05 were considered statistically significant, and differences between samples within a group were evaluated using a Student's t-test (p<0.05).

While illustrative embodiments have been illustrated and described, it will be appreciated that various changes can be made therein without departing from the spirit and scope of the invention.

1. A porous chitosan scaffold, comprising chitosan and having a compressive modulus from about 3 to about 20 MPa and compressive strength from about 0.2 to about 2.0 MPa, wherein the scaffold comprises pores having pore walls.

2. A porous chitosan scaffold, consisting essentially of chitosan, wherein the scaffold comprises pores having pore walls.

3. A porous chitosan scaffold, consisting of chitosan, wherein the scaffold comprises pores having pore walls.

4. The scaffold of claim 1 having a porosity of from about 88 to about 98 percent.

5. The scaffold of claim 1 having a pore size distribution from about 30 to about 500 μm.

6. The scaffold of claim 1, wherein the pore walls have a thickness from about 12 to about 50 μm.

7-8. (canceled)

9. The scaffold of claim 1 further comprising an additive for supporting cell growth.

10. The scaffold of claim 1 further comprising cells.

11. The scaffold of claim 2 having a compressive modulus from about 3 to about 20 MPa and compressive strength from about 0.2 to about 2.0 MPa.

12. A method for expanding a population of cells, comprising:
(a) seeding a porous scaffold of claim 1 with cells; and
(b) culturing the cells in the presence of the scaffold to provide a scaffold populated with the cells.

13. A method for tissue engineering, comprising introducing the scaffold of claim 1 into a tissue to be engineered.

14. A method for making a porous chitosan scaffold, comprising:
(a) combining chitosan and an aqueous acid solution to provide a first aqueous chitosan solution having a first viscosity;
(b) conditioning the first aqueous chitosan solution to provide a second aqueous chitosan solution having a second viscosity, wherein the second viscosity is greater than the first viscosity, and wherein the second aqueous chitosan solution does not include an organic solvent;
(c) freezing the second aqueous chitosan solution to provide a frozen chitosan composition; and
(d) freeze-drying the frozen chitosan composition to provide a porous chitosan scaffold.

15. A method for making a porous chitosan scaffold, consisting essentially of:
(a) combining chitosan and an aqueous acid solution to provide a first aqueous chitosan solution having a first viscosity;
(b) conditioning the first aqueous chitosan solution to provide a second aqueous chitosan solution having a second viscosity, wherein the second viscosity is greater than the first viscosity;
(c) freezing the second aqueous chitosan solution to provide a frozen chitosan composition; and
(d) freeze-drying the frozen chitosan composition to provide a porous chitosan scaffold.

16-37. (canceled)


SEQUENCE LISTING (14 sequences)

SEQ ID NO: 1 - DNA, 16 bp, Artificial Sequence
  Synthetic
aaagcccagcgactct

SEQ ID NO: 2 - DNA, 21 bp, Artificial Sequence
  Synthetic
ctaaacggtggtgccatagat

SEQ ID NO: 3 - DNA, 21 bp, Artificial Sequence
  Synthetic
acaagctccacctggactaca

SEQ ID NO: 4 - DNA, 19 bp, Artificial Sequence
  Synthetic
tcttcttcacacgcagttt

SEQ ID NO: 5 - DNA, 19 bp, Artificial Sequence
  Synthetic
tcctgccgatgtcgctatc

SEQ ID NO: 6 - DNA, 21 bp, Artificial Sequence
  Synthetic
caagttccggtgtgactcgtg

SEQ ID NO: 7 - DNA, 20 bp, Artificial Sequence
  Synthetic
cagaggaggcaagcgtcact

SEQ ID NO: 8 - DNA, 20 bp, Artificial Sequence
  Synthetic
ctgtctgggtgccaacactg

SEQ ID NO: 9 - DNA, 20 bp, Artificial Sequence
  Synthetic
gcttctccaacccacgaatg

SEQ ID NO: 10 - DNA, 21 bp, Artificial Sequence
  Synthetic
gaactgataggacgctgacga

SEQ ID NO: 11 - DNA, 24 bp, Artificial Sequence
  Synthetic
tcgcctaccagctcatgcataaca

SEQ ID NO: 12 - DNA, 24 bp, Artificial Sequence
  Synthetic
tgaagctcttccaggtgtcaacga

SEQ ID NO: 13 - DNA, 21 bp, Artificial Sequence
  Synthetic
caggattccatacccaagaag

SEQ ID NO: 14 - DNA, 19 bp, Artificial Sequence
  Synthetic
aaccctaaggccaaccgtg